(19) United States
(12) Patent Application Publication
(10) Pub. No.: US 2009/0301499 A1
(43) Pub. Date: Dec. 10, 2009

(54) Device for alleviating snoring

(76) Inventors: Robert Chalk, Enfield Middx (JP); Chris Grant, Essex (GB)

Correspondence Address: WILEY HORTON, 215 SOUTH MONROE STREET, 2ND FLOOR, TALLAHASSEE, FL, 32301, US

(21) Appl. No.: 12/308,156

(22) PCT Filed: Jan. 6, 2007
(86) PCT No.: PCT/GB2007/002027

§ 371 Date: Jul. 7, 2009

(30) Foreign Application Priority Data
Jun. 6, 2006 (GB) 0611132.2
Dec. 5, 2006 (GB) 0624306.7

(51) Int. Cl. A61F 5/56

(52) U.S. Cl. 128/848

(57) ABSTRACT

A device for alleviating snoring 1 comprising first and second substantially spherical parts, 2, 3, the first and second spherical parts 2,3 being insertable into a first and a second nasal passage respectively of a user, wherein, in use, air flow through the nasal passages of the user is substantially restricted.

### CROSS-REFERENCES TO RELATED APPLICATIONS

This is a U.S. National Phase filing made under 35 U.S.C. §371. This application claims the benefit of an earlier-filed PCT application having serial number PCT/GB2007/002027. The PCT application listed the same inventors and specified a priority date of 6 Jun. 2006.

### STATEMENT REGARDING FEDERALLY SPONSORED RESEARCH OR DEVELOPMENT

Not Applicable.

### MICROFICHE APPENDIX

Not Applicable.

### BACKGROUND OF THE INVENTION

1. Field of the Invention

This invention relates to the field of human sleep. More specifically, the invention comprises a device which seeks to reduce the occurrence and/or the intensity of snoring.

2. Description of the Related Art

Snoring often results as a consequence of breathing through the open mouth such that air flow causes the uvula and soft palate to vibrate producing an often harsh sound. The resulting sound can often be of such a level that it disturbs the sleep of others.

A number of devices for alleviating snoring are known, these include nasal strips which are affixed to the skin on the bridge of the nose and seek to maintain an unrestricted air flow though the nostril into the nasal cavity.

Other devices for alleviating snoring such as that disclosed in EP 0 824 015 include devices which are inserted into the nose and seek to open and shape the nasal passages so that breathing is facilitated.

Further examples of devices to alleviate snoring include gum-shield type devices such as that disclosed in WO 98/02119, which encourage the wearer to breathe through the nose.

However, these inventions can be cumbersome and uncomfortable.

The present invention seeks to overcome these disadvantages and provide a new and inventive approach to reducing the occurrence of snoring, which in contrast to the prior art, where it is taught to increase the air flow through the nasal passages, the present invention restricts the air flow through the nasal passages.

### BRIEF SUMMARY OF THE INVENTION

According to the present invention there is provided a device for alleviating snoring integrally formed of a resilient material and comprising first and second generally spherical parts connected by a bridge part, the first and second spherical parts being insertable into a first and a second nasal passage respectively of a user, wherein in use air flow through the nasal passages of the user is substantially restricted.

In order to insert the spherical parts of the device in to the nasal passages of a user, the device must be inserted through the user's nostrils. The irregular shape and size of the user's nostrils may make the insertion of the device difficult.

In the present device, the spherical parts are made of a deformable material, such that the user may deform the parts to aid their insertion through the nostril. Although the spherical parts may be deformed, the overall volume or size of the parts remains unchanged and it may still prove difficult to insert the device into the nasal passage of the user. An embodiment of the present invention seeks to overcome the aforementioned problem.

Preferably the first and second generally spherical parts are hollow and have an opening in the peripheral wall thereof.

Preferably the spherical parts may be provided with internal support members, which may be equiangular spaced webs.

Preferably, the resilient material has delayed resilience so that a user may deform the material and insert the spherical parts into the nostrils, before the material returns substantially to its original form.

Preferably, the resilient material is a plastic material. The plastic material may be a hard plastic material or a soft plastic material. A soft plastic material will provide a more comfortable fit against the soft and often sensitive inner surface of the wearer's nose.

Preferably, the plastics material is a pharmaceutical grade plastic which is smooth and easy to handle. The device may also be made of a plastic material such as rubber, latex, silicone or a combination thereof.

Preferably, the plastics material is transparent or flesh colored. This enables a user to use the device discreetly.

Preferably, the spherical parts are formed to a generally acorn or nipple shaped end, which seeks to provide improved comfort and fit inside the nostril of the user.

The bridge part is preferably circular in cross section and formed of the same material as the spherical parts. In this way, the device, including the bridge part, is formed of one piece. The bridge part may be used to assist in the removal of the device from the user's nose. The bridge part may also be formed to provide a loop or grip for the user's fingers.

In a further preferred embodiment, a nose clip is provided which in use is positioned substantially adjacent to the external rear part of the nose at the junction between the nostrils and the face and seeks to provide an improved seal between the device and the wall of the nasal passage. The nose clip is substantially U-shaped in form. The nose clip may be joined to the bridge of the device. The nose clip has a degree of resilience such that it pinches the nostril closer to the spherical part to a ensure a closer fit of the spherical part in the nasal cavity. The clip may be covered in a soft material to provide a comfortable fit against the user's nose.

### BRIEF DESCRIPTION OF THE SEVERAL VIEWS OF THE DRAWINGS

FIG. 1 is a front view of the device;

FIG. 2 is a top view of the device;

FIG. 3 is a view of the device in use;

FIG. 4 is a view of the device in use with a nasal clip;

FIG. 5 is a top view of the device wherein the generally spherical parts are hollow;

FIG. 6 is a side view of the device shown in FIG. 5;

FIG. 7 is a bottom view of the device shown in FIG. 5.

### DETAILED DESCRIPTION OF THE INVENTION

FIG. 1 shows a front view of the device, 1, according to a preferred embodiment of the present invention. Generally spherical parts 2, 3 are made of a soft resilient material with a smooth external surface. The material may be a plastic material such as a pharmaceutical grade plastic which is smooth and easy to handle. The material used to form the generally spherical parts may also comprise rubber, latex, silicon or a combination thereof. The device 1 may have a further outer layer or a surface coating or a lubricant. This coating or lubricant may serve to facilitate the insertion of the device, The coating may also serve to prevent a reaction by a user who is allergic to latex materials. The device is designed so that the first and second generally spherical parts fit into the respective vestibule areas of the user's nose and substantially fill the lower part of the nostril so that air flow through the nose is substantially restricted. The user is then forced to breath through the mouth. The two generally spherical parts 2, 3 are attached to each other by a substantially U-shaped loop 4. This loop is formed of the same material as the spherical parts and in use, fits under the columella of the user's nose.

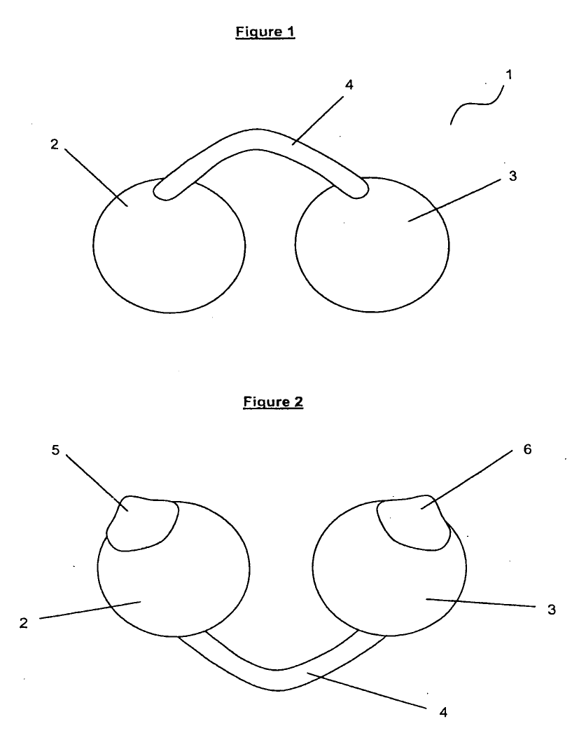

FIG. 2 is a top view of the device 1 and shows a nipple shaped form, 5, 6. The nipple shaped form is a protuberance from the surface of the generally spherital part 2, 3. The nipple form 5, 6 is located on the upper edge of the generally spherical part, so that the nipple shaped form 5, 6 is positioned uppermost in the user's nose, when the device 1 is inserted into the nasal passage of the user. This nipple form 5, 6 seeks to provide an improved fit in the nasal passage of the user and also seeks to aid the location of the device in the nose. The nipple shaped form 5, 6 also seeks to facilitate the insertion of the device by the user. The nipple shaped form 5, 6 may also be formed of the same material as the generally spherical parts 2, 3 and the device comprising the nipple shaped form 5, 6 may therefore be formed as one piece. The nipple shaped form 5, 6 may be of a different hardness to the generally spherical part 2, 3 and may be formed of an alternative material and joined to the generally spherical part 2, 3 using for example an adhesive.

FIG. 3 is a view of the device in use. The device is located in the lower nasal cavity of the user's nose 7. The bridge part 4 is seen resting under the columella 8 of the user's nose 7. In use, the generally spherical parts 2, 3 do not protrude substantially from the nostrils of the user. To facilitate the discreet wearing of the device, the bridge part 4 may be formed of a transparent or flesh colored material.

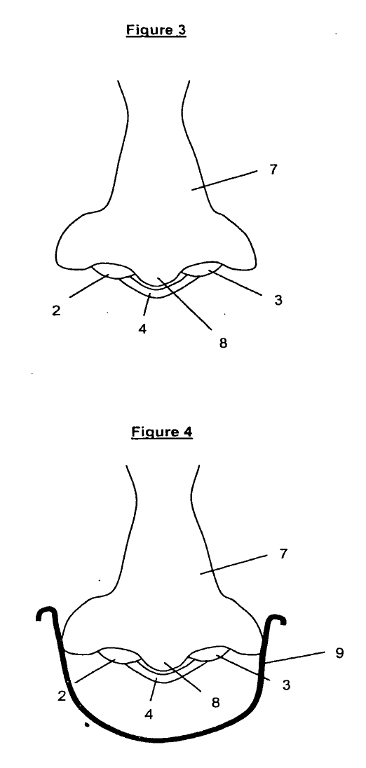

FIG. 4 is a view of the device in use with the nose clip 9. The nose clip 9 is substantially U-shaped in form. The nose clip 9 may be formed of a plastic or a metal or a combination thereof. The nasal clip 9 has a degree of resilience such that it pinches the nostril closer to the spherical part 2, 3 to a ensure a closer fit of the spherical part 2, 3 in the nasal cavity. The nose clip 9 is positioned substantially adjacent to the external rear part of the nose 7 at the junction between the nostrils and the face and seeks to provide an improved seal between the device 1 and the wall of the nasal passage. The nose clip 9 may comprise two small loops at each end of the clip 9 such that in use these loops rest against the junction between the nostrils and the face. The clip may be covered in a soft material to provide a comfortable fit against the user's nose 7. The nose clip may be joined to the bridge part 4 of the device 1 using fixing means. The fixing means may for example include a hole in the bridge part 4 through which the clip 9 is located or adhesive or a loop and hook type means.

FIG. 5 shows a top view of the device, 11, according to a preferred embodiment of the present invention. Generally spherical parts 12, 13 are made of a soft resilient material. The material may be a plastics material such as a pharmaceutical grade plastic which is smooth and easy to handle. The material used to form the generally spherical parts may also comprise rubber, latex, silicon or a combination thereof.

The device 11 may have a further outer layer or a surface coating or a lubricant. This coating or lubricant may serve to facilitate the insertion of the device. The coating may also serve to prevent a reaction by a user who is allergic to latex materials. The device is designed so that the first and second generally spherical parts 12, 13 fit into the respective vestibule areas of the user's nose and substantially fill the lower part of the nostril so that air flow through the nose is substantially restricted. The user is then forced to breath through the mouth. The two generally spherical parts 12, 13 are attached to each other with a substantially U-shaped loop or bridge part 14. This loop is formed of the same material as the spherical parts and in use, fits under the columella of the user's nose. To facilitate the discreet wearing of the device, the bridge part 14 may be formed of a transparent or flesh colored material.

Generally acorn or nipple shaped forms 15, 16 are formed as protuberances from the surface of the generally spherical parts 12, 13. The nipple shaped forms 15, 16 are located on the upper edge of the generally spherical parts, so that the nipple shaped forms 15, 16 are positioned uppermost in the user's nose, when the device 11 is inserted into the nasal passage of the user. These nipple shaped forms 15, 16 seek to provide an improved fit in the nasal passage of the user and also seek to aid the location of the device in the nose. The nipple shaped forms 15, 16 also seek to facilitate the insertion of the device by the user. The nipple shaped forms 15, 16 may also be formed of the same material as the generally spherical parts 12, 13 and the device comprising the nipple shaped forms 15, 16 may therefore be formed as one piece. The nipple shaped forms 15, 16 may be of a different hardness to the generally spherical part 12, 13 and may be formed of an alternative material and joined to the generally spherical part 12, 13 using for example an adhesive.

In the embodiment shown, the spherical parts 12, 13 are each provided with an opening in the peripheral walls thereof in the form of holes 20,30. The holes 20, 30 are positioned at right angles to the point where the bridge part 14 joins each of the spherical parts 12, 13. The holes 20, 30 pass through the outer peripheral wall of the spherical parts 12, 13 to the hollow centres. The spherical parts 12, 13 are provided with internal support members 21, 22 in the hollow centre of the spherical parts 12, 13 which serve to return the device to its normal shape after deformation.

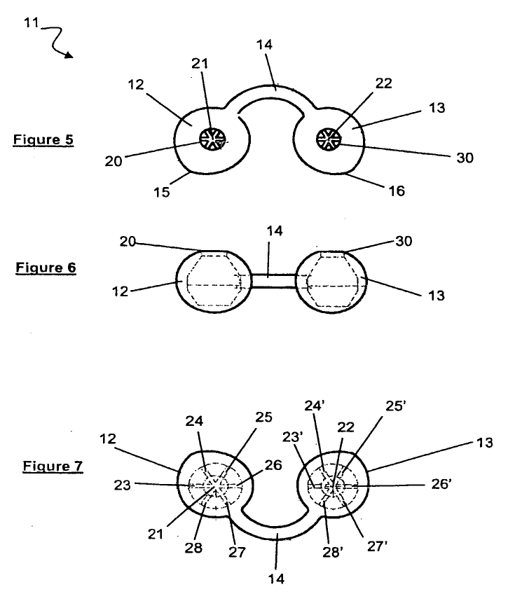

FIG. 6 is a side view of the device 11. The outlines of the hollow centres of the spherical parts 12, 13 are shown as dashed lines. The profile of the hollow centre generally follows the shape of outer surface profile of the spherical parts 12, 13.

FIG. 7 is a bottom view of the device. The outline of the hollow centres of the spherical parts 12, 13 are shown as dashed lines. Each of the internal support members 21, 22 comprise six equiangular spaced webs 23, 24, 25, 26, 27, 28 and 23′, 24′, 25′, 26′, 27′, 28′ which meet at their ends at the centre of the spherical parts 12, 13 at a point in line with the axis of the holes 20, 30 respectively. The webs 23, 24, 25, 26, 27, 28 and 23′, 24′, 25′, 26′, 27′, 28′ are formed as longitudinal rectangular cross-sectional members. The height of the webs 23, 24, 25, 26, 27, 28 and 23′, 24′, 25′, 26′, 27′, 28′ is approximately a third the diameter of the spherical parts 12, 13. The webs 23, 24, 25, 26, 27, 28 and 23′, 24′, 25′, 26′, 27′, 28′ are located in the bottom third of the spherical parts 12, 13 away from the holes 20, 30.

The members 21, 22 are formed of the same material as the spherical parts 12,13 and formed as one piece therewith. When the spherical parts 12, 13 are deformed by the user's fingers, the webs 23, 24, 25 and 26, 27, 28 buckle, reducing the overall size of the spherical parts 12, 13 which facilitates insertion of the device 11 through the nostril of the user into the user's nasal passages. Once the spherical parts 12, 13 have been inserted in the user's nasal passages, the webs de-buckle and return the spherical parts 12, 13 substantially to their original or former form.

In an alternative application, the device may be worn by a user whilst swimming. Since the device effectively blocks the nasal passages it serves to substantially reduce or eliminate the occurrence of the ingression of water into the user's nose. In this form, the spherical parts have no or a very small opening and may be filled with a gel.

What is claimed is:

1. A device for alleviating snoring integrally formed of a resilient material and comprising first and second generally spherical parts connected by a bridge part, the first and second spherical parts being insertable into a first and a second nasal passage respectively of a user, wherein, in use, air flow through the nasal passages of the user is substantially restricted.

2. A device for alleviating snoring according to claim 1, wherein the said parts are hollow and have an opening in the peripheral wall thereof.

3. A device for alleviating snoring according to claim 2, wherein the spherical parts may be provided with internal support members.

4. A device for alleviating snoring according to claim 2, wherein the internal support members comprise equiangular spaced webs.

5. A device for alleviating snoring according to claim 1, wherein the first and second spherical parts have a smooth outer surface.

6. A device for alleviating snoring according to claim 1, wherein the spherical parts have an outer coating.

7. A device for alleviating snoring according to claim 1, wherein the device includes material selected from the group consisting of rubber, latex, and silicone.

9. A device for alleviating snoring according to claim 1, wherein the spherical parts are formed to a nipple shaped end.

10. A device for alleviating snoring according to claim 1, wherein the device further includes a nasal clip.

11. A device for alleviating snoring according to claim 2, wherein the device includes material selected from the group consisting of rubber, latex, and silicone.

12. A device for alleviating snoring according to claim 3, wherein the internal support members comprise equiangular spaced webs.

13. A device for alleviating snoring according to claim 2, wherein the first and second spherical parts have a smooth outer surface.

14. A device for alleviating snoring according to claim 3, wherein the first and second spherical parts have a smooth outer surface.

15. A device for alleviating snoring according to claim 4, wherein the first and second spherical parts have a smooth outer surface.

16. A device for alleviating snoring according to claim 2, wherein the spherical parts have an outer coating.

17. A device for alleviating snoring according to claim 3, wherein the spherical parts have an outer coating.

18. A device for alleviating snoring according to claim 4, wherein the spherical parts have an outer coating.

19. A device for alleviating snoring according to claim 3, wherein the device includes material selected from the group consisting of rubber, latex, and silicone.

20. A device for alleviating snoring according to claim 4, wherein the device includes material selected from the group consisting of rubber, latex, and silicone.
